(19) United States
(12) Patent Application Publication
(10) Pub. No.: US 2024/0360401 A1
(43) Pub. Date: Oct. 31, 2024

(54) METHOD FOR RELEASING PLATELETS IN TURBULENT FLOW AND PLATELET RELEASE SYSTEM FOR CARRYING OUT SUCH METHOD

(71) Applicants: ETABLISSEMENT FRANÇAIS DU SANG, SAINT DENIS (FR); INSTITUT NATIONAL DE LA SANTE ET DE LA RECHERCHE MEDICALE (INSERM), PARIS (FR); UNIVERSITE DE STRASBOURG, STRASBOURG (FR); AVIGNON UNIVERSITE, AVIGNON (FR); CENTRE NATIONAL DE LA RECHERCHE SCIENTIFIQUE, PARIS (FR); UNIVERSITE D'AIX MARSEILLE, MARSEILLE (FR); ECOLE CENTRALE DE MARSEILLE, MARSEILLE (FR)

(72) Inventors: Catherine STRASSEL, STRASBOURG (FR); Léa MALLO, STRASBOURG (FR); Valentin DO SACRAMENTO, URMATT (FR); Eric BERTRAND, MARSEILLE (FR); Yannick KNAPP, MARSEILLE (FR)

(21) Appl. No.: 18/287,728

(22) PCT Filed: Apr. 21, 2022
(86) PCT No.: PCT/EP2022/060554

§ 371 Date: Oct. 20, 2023

(30) Foreign Application Priority Data
Apr. 21, 2021 (FR) 2104123

(51) Int. Cl. C12M 1/00; C12M 3/04

(52) U.S. Cl. CPC C12M 47/04; C12M 23/34; C12M 27/10

(57) ABSTRACT

The disclosure relates to a method for releasing platelets from megakaryocytes contained in a fluid (F), the method being carried out by means of a system comprising two concentric cylinders, an inner cylinder comprising a cylindrical wall and a hollow outer cylinder located radially external to the inner cylinder, the outer cylinder comprising a cylindrical wall of a base at which the second cylinder is closed, the cylinders being separated by a space devoid of any mechanical parts, the space being intended to receive the fluid (F), the method comprising the following steps: supplying the space with a fluid (F) comprising megakaryocytes, rotating the inner cylinder about its axis, the outer cylinder being stationary, or moving the two cylinders in opposite directions about their axes, so as to generate an at least partially turbulent flow of fluid (F) in the space and obtain a second fluid (F′) enriched in platelets.

### TECHNICAL FIELD OF THE INVENTION

The invention relates to a method for releasing platelets from a fluid comprising, in particular, megakaryocyte progenitors. The method according to the invention is implemented by means of a platelet release system designed for this purpose. The invention also relates to such a system for releasing platelets. The invention is particularly suited to the in vitro production of blood platelets on an industrial scale and to the production of extracellular vesicles.

### TECHNICAL BACKGROUND

Until now, the usual solution for implementing in vitro platelet release methods using small systems—typically from a few tens of micrometres to a few centimetres—has been to reproduce the natural mechanisms for releasing platelets occurring in the human body. For this reason, these methods are known as biomimetic.

The mechanisms leading to the release of platelets occurring in the human body are still the subject of much research. We know that platelet release occurs during a process of fragmentation of the megakaryocytes and/or of the cytoplasmic extensions into platelets. This highly coordinated in vivo process occurs naturally in the blood as the cytoplasmic extensions pass through the endothelial barrier and then the proplatelets into the pulmonary microcirculation as they mature, thanks to the force of the blood flow. The megakaryocytes play an essential role as precursor cells. These mechanisms operate at very slow speeds in the human body, and it is also at relatively slow speeds that platelets production is performed in the existing biomimetic microfluidic reactors.

The document WO2014107240A1 proposed microfluidic bioreactors comprising structures comprising microperforated walls which, when the fluid circulates through them, simulate the endothelial passage. In normal operation, the fluid flow regime is laminar and the shear rates measured at the level of the microperforated walls correspond to physiological shear rates. The treatment times are generally several hours. The same applies to the document WO2018165308 A1, in which a membrane plays the role of the microperforated wall of the document WO2014107240A1.

Recently, efforts have been made to industrialise the method for releasing platelets and new types of bioreactor have been developed. The document WO201909364A1 discloses an example of a bioreactor intended for this type of use. The proposed system aims to increase the number and service life of healthy platelets. It comprises a reservoir for the fluid comprising megakaryocytes and at least one means for generating a turbulent flow within the reservoir so as to allow platelets to be released. In one embodiment, the means consists of a blade moving back and forth along the reservoir. The disadvantage of such a system lies in the fact that the blade is likely to collide with the megakaryocytes contained in the fluid, with the result that a significant proportion of the megakaryocyte precursors obtained during the use of the system are damaged. In fact, the presence of the blades in the volume in which the fluid is moving is already responsible for collisions with the megakaryocyte precursors. Secondly, the movement of the blades is likely to generate higher intensity impacts with the megakaryocyte precursors. A significant proportion of the platelets obtained therefore have significantly reduced or even non-existent activity. In addition, the cover la of the culture/release reservoir allows the axle to pass through, allowing the blade to move back and forth. Such an arrangement is likely to prevent sterile conditions from being maintained in the reservoir.

Document EP3372674A1 (equivalent to document WO2017/077964A1) discloses another method for releasing platelets from megakaryocytes, this method being implemented by the team of document WO201909364A1. The bioreactor comprises a blade that moves back and forth up and down so as to create agitation in a culture fluid comprising megakaryocytes. As with the method described in document WO201909364A1, the presence of a blade moving in the space where the fluid from which platelet release is carried out circulates, is likely to generate impacts with the megakaryocyte precursors and thus reduce the activity of the platelets released from said megakaryocyte precursors.

The invention aims to overcome the aforementioned disadvantages and to this end proposes a method for releasing platelets from megakaryocytes contained in a fluid, said method being implemented by means of a system comprising two concentric cylinders, an inner cylinder comprising a cylindrical wall and a hollow outer cylinder located radially external to the inner cylinder, said outer cylinder comprising a cylindrical wall of a base at the level of which said second cylinder is closed, said cylinders being separated by a space devoid of any mechanical part, said space being intended to receive the fluid, said method comprising the following steps:
(100) supplying said space with a fluid comprising megakaryocytes,
(200) moving the inner cylinder in rotation about its axis, the outer cylinder being stationary, or moving the two cylinders in opposite directions about their axes, so as to generate an at least partially turbulent fluid flow in said space and obtain a second fluid enriched with platelets.

The method for releasing the platelets in turbulent regime used in this invention allows to prevent any degradation of the platelets thanks to the structure of its platelet release system, thus significantly improving the platelet release efficiency and foreshadowing an industrial method. The concentric cylinders of the system used in the method according to the invention are separated by a space devoid of any mechanical parts so that when the cylinder or cylinders move around their axis, the platelets are released into the fluid without any other part or element of the system located in the inter-cylinder space being able to impact them.

In addition, as the cylinders are concentric, this guarantees a constant air gap along the walls. The air gap is the distance between the two walls. It is an operating parameter that can be manipulated to better control the flow regime in the system to allow the release of the platelets. At the same time, the value of the air gap itself can allow to further improve this efficiency of platelet release. It is also possible to influence the speed of movement of the cylinder or cylinders to obtain the conditions of a turbulent flow for the fluid. In this way, when the method is implemented, the operating parameters can be adjusted to obtain the conditions specific to the turbulent flow regime.

According to various characteristics of the invention taken together or separately:
when the inner cylinder is moved while the outer cylinder is stationary, the inner cylinder is moved so as to define a Reynolds number

$${Re}_{i} = \frac{{r}_{i} \cdot {ω}_{i} \cdot d}{ϑ}$$

greater than or equal to 1400, where rᵢ is the radius of the inner cylinder, ωᵢ is the angular speed of the inner cylinder, d is a distance separating the inner cylinder from the outer cylinder and ϑ is the kinematic viscosity of the fluid between said inner and outer cylinders;
when the two cylinders are moved in opposite directions, the inner cylinder and the outer cylinder are moved so as to respectively define a Reynolds number

$${Re}_{i} = \frac{{r}_{i} \cdot {ω}_{i} \cdot d}{ϑ}$$

greater than 1000 and a Reynolds number

$${Re}_{o} = \frac{{r}_{o} \cdot {ω}_{o} \cdot d}{ϑ}$$

greater than 1000, where rᵢ is the radius of the inner cylinder, rₒ is the radius of the outer cylinder, ωᵢ is the angular speed of the inner cylinder, ωₒ is the angular speed of the outer cylinder, d is a distance separating the inner cylinder from the outer cylinder and ϑ is the kinematic viscosity of the fluid between said inner and outer cylinders;
the two cylinders are separated by a distance d of less than 5 mm;
the two cylinders are separated by a distance d of between 2 mm and 4 mm;
the two cylinders are separated by a distance d of approximately 3 mm;
in step 100, the space is continuously supplied with the fluid, the system comprising an inlet for filling the space with said fluid and an opening for evacuating the platelet-enriched fluid, said opening being located at the level of the base;
during step 200, the residence time of the fluid in said space is between 4 minutes and 6 minutes, preferably about 5 minutes;
the inner cylinder and the outer cylinder comprise other peripheral internal walls forming a first pattern and a second pattern respectively, said first and second patterns being nested in one another so that said other walls of the inner cylinder and other walls of the outer cylinder are concentric with one another and at least partially opposite one another;
method in which extracellular vesicles are also released.

According to an alternative embodiment of the present invention, the method concerns a method for releasing platelets from megakaryocytes contained in a fluid, said method being implemented by means of a system comprising two parallel flat walls separated by a space devoid of any mechanical part, said space being intended to receive the fluid, said walls being able to move, said method comprising the following steps:
(100) supplying said with a fluid space comprising megakaryocytes,
(200) moving one of said flat walls in a plane of said flat wall, the other of said walls being stationary, or moving the two walls in opposite directions, each wall being moved in its plane, so as to generate an at least partially turbulent fluid flow in said space and obtain a second fluid enriched with platelets.

According to various characteristics of the invention taken together or separately:
the first flat wall is the wall of a first conveyor belt and the second flat wall is the wall of a second conveyor belt;
when the two walls move in opposite directions, the two walls move in opposite directions at the same speed;
the two walls move at a speed of about 1 metre per second;
when the two walls are moving in opposite directions, wherein the two walls move with a speed difference of at most 10% with respect to an average value;
extracellular vesicles are also released.

### BRIEF DESCRIPTION OF THE FIGURES

FIG. 1a illustrates schematically, in perspective, a platelet release system allowing to implement the method according to a first embodiment of the invention;

FIG. 1b illustrates schematically, in cross-section, a platelet release system allowing to implement the method according to a first embodiment of the invention;

FIG. 2a schematically illustrates, in top view, the platelet release system of FIG. 1 with the profile of the speeds between the inner cylinder and the outer cylinder, as both cylinders rotate. The lower figure shows the radial speed profile of the fluid in the space;

FIG. 2b schematically illustrates, in top view, the platelet release system of FIG. 1 with the profile of the speeds between the inner cylinder and the outer cylinder, as the inner cylinder rotates. The lower figure shows the radial speed profile of the fluid in the space;

FIG. 3a illustrates schematically, in perspective, an alternative embodiment of the platelet release system illustrated in FIG. 1;

FIG. 3b schematically illustrates, in cross-section, an alternative embodiment of the platelet release system illustrated in FIG. 1;

FIG. 4a illustrates schematically, in perspective, a platelet release system allowing to implement the method according to a second embodiment of the invention;

FIG. 4b schematically illustrates, in cross-section, a platelet release system allowing to implement the method according to a second embodiment of the invention;

FIG. 5a schematically illustrates an alternative embodiment of the platelet release system illustrated in FIGS. 4a and 4b;

FIG. 5b schematically illustrates an alternative embodiment of the platelet release system illustrated in FIGS. 4a and 4b;

FIG. 6 illustrates Andereck's diagram (1986);

FIG. 7 illustrates the number of double-positive platelets for a reference sample, for a sample treated with the method according to the invention with a system for releasing platelets according to the embodiment illustrated in FIGS. 1a and 1b with an air gap of 3 mm, 4 mm and 5 mm (from left to right), the cylinders rotating in opposite directions;

FIG. 8 illustrates the number of double-positive platelets for a reference sample (left-hand point), for a sample treated with the method according to the invention with a system for releasing platelets as illustrated in FIGS. 1a and 1b with a 3 mm air gap, the cylinders rotating in opposite directions (central point), the inner cylinder being the only one to rotate (right-hand point);

FIG. 9 is an example of a two-cylinder device for implementing the method of the present;

FIG. 10 illustrates the number of CD41+ and CD42+ platelets released for a) the passage of 5,000 beads in flow cytometry, b) for the number of platelets per cell on day 7 of culture, and c) for the number of platelets per CD34+ seeded, with the white columns representing the results obtained with a pipette, the grey columns representing the results obtained in a configuration where the two cylinders rotate in opposite directions, and the black columns representing the results obtained in a configuration where only the inner cylinder rotates;

FIG. 11 shows the number of platelets expressing the major glycoproteins on their surface, depending on the mode of release. FIG. 11a compares the percentage of positive platelets between a configuration where the two cylinders rotate in opposite directions (grey columns) and a configuration where only the inner cylinder rotates (black columns). FIG. 11b shows a comparison between a configuration where the two cylinders rotate in opposite directions (grey columns) and the use of a pipette (white columns). FIG. 11c shows a comparison between a configuration in which only the inner cylinder rotates (black columns) and the use of a pipette (white columns);

FIG. 12 illustrates the pre-activated (resting) state of the released platelets and their ability to activate when placed in the presence of an antagonist (thrombin), with the white columns representing the results obtained with a pipette, the grey columns representing the results obtained in a configuration where the two cylinders rotate in opposite directions and the black columns representing the results obtained in a configuration where only the inner cylinder rotates. The figure on the left corresponds to the results obtained by analysing a GPIIb-IIIa pre-activation marker, while the figure on the right corresponds to the results obtained by analysing the expression of P-selectin on the surface of the platelets;

FIG. 13 illustrates the recirculation phenomenon of the platelets (ratio of the number of platelets to the number of platelets at 3 minutes as a function of time) for cultured platelets in a murine organism obtained with the method of the invention in the case where only the inner cylinder rotates (left figure, squares) compared to a mixture of platelet concentrate (left figure, solid circles) and cultured platelets obtained using the method of the invention in the case where the two cylinders rotate in opposite directions (right figure, squares) compared to a mixture of platelet concentrate (right figure, solid circles);

FIG. 14 shows immunofluorescence images of the platelets released by manual pipetting (FIG. 14a)), with the method of the invention in the case where only the inner cylinder rotates (FIG. 14b)), with the method of the invention in the case where the two cylinders rotate in opposite directions.

### DETAILED DESCRIPTION OF THE INVENTION

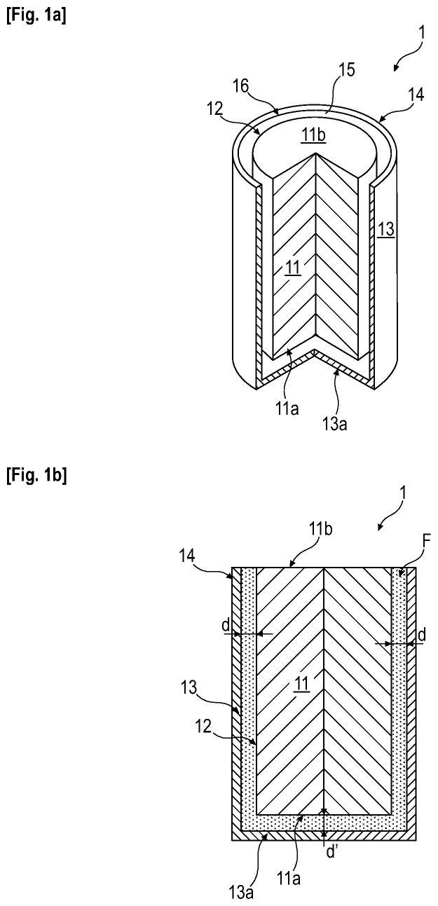

The invention relates to a method for releasing platelets from a fluid F comprising megakaryocytes and a system 1, as exemplified in FIGS. 1a, 1b, 2a, 2b, 3a, 3b, 4a, 4b, 5a and 5b allowing to carry out said method.

The fluid F in question is, for example, a culture medium containing a population of cells obtained from immortalized or non-immortalized strains cells at different stages of differentiation, including megakaryocyte progenitors, megakaryocytes. The megakaryocytes or megakaryocytic cells are large blood cells (up to 100 μm and 30 μm in culture) which, when mature, have long extensions called cytoplasmic extensions or proplatelets.

The mechanisms involved in the formation of the blood platelets are still the subject of much research. Among them, the platelet release occurs during a process of fragmentation of the Mk megakaryocytes and/or cytoplasmic extensions, Ck, into platelets. This is a highly coordinated in vivo process that occurs naturally in the blood thanks to the force of the blood flow. The megakaryocytes Mk play an essential role as they are precursor cells. However, the process of release of the platelets in the body remains poorly described and there are still many questions about transendothelial passage and the precise role of the blood flow in the formation of the platelets. This process was reproduced in vitro using microfluidic systems, allowing certain in vivo mechanisms to be corroborated by microfluidic experiments. This process can also be reproduced in vitro using a manual pipette or using devices such as those presented in the introductory part of this description, which also allows to provide a better understanding of the mechanisms involved in the release of the platelets (Strassel et al. “Aryl hydrocarbon receptor-dependent enrichment of a high potential to produce propalets”, Blood, 5 May 2016, vol. 127, no 18). The production of platelets in vitro allows to provide a better understanding of the mechanisms involved in the formation of the platelets. However, the release efficiency of the platelets are generally lower than those obtained in vivo. The invention thus aims to reproduce the fragmentation process of the Mk megakaryocytes and/or of the Ck cytoplasmic extensions of method for releasing platelets under a turbulent regime.

Before describing the invention in more detail, it should be noted that in addition to allowing the release of the platelets, the invention also allows other functional cytoplasmic elements, such as extracellular vesicles, to be released from the platelets thus released. The extracellular vesicles are now recognised as vectors of biological material capable of transferring this content between cells. The extracellular vesicles are nanovesicles derived from cell membranes and physiologically secreted into the extracellular medium. They are made up of several subtypes (Exosomes, Microvesicles, Apoptotic bodies) and vary in size from 30 nm to 1 μm. By way of comparison, the platelets are generally between 3.5 μm and 5 μm in diameter.

The principles behind this method for releasing the platelets are presented in the following section. While there are many ways of generating a turbulent flow, the method described in this invention uses mechanisms similar to those used to generate instabilities in a system of two concentric circular cylinders. These mechanisms, which underlie the operation of the turbomachines and the formation of the planets, are generally used in large-scale systems. It should be remembered that in the technical field of the present invention, the prevailing trend is to use small-scale systems as described in the introductory part of the description, even though more and more pre-industrial bioreactors are being implemented.

See the publication by C. Andereck et al. “Flow regimes in a circular Couette system with independently rotating cylinders”, J. Fluid Mech. 1986, and the publication of Grossmann et al. “High-Reynolds Number Taylor-Couette Turbulence”, Annu. Rev. Fluid Mech., 2016. In the following, we will focus more specifically on the phase diagram of Andereck et al. which is illustrated in these publications and which is reproduced in FIG. 6 of this application. The phase diagram of Andereck et al. illustrates the different flow regimes that can be generated in a system of two concentric circular cylinders whose angular speed and therefore Reynolds number can be varied to generate turbulence. The Andereck phase diagram shows the Reynolds number of the inner cylinder (Reᵢ or Rᵢ depending on the publication) on the abscissa and the Reynolds number of the outer cylinder (Reₒ or Rₒ depending on the publication) on the ordinate. It thus defines the flow regimes according to the aforementioned parameters, so that each type of flow is characterised by given pairs (Reᵢ, Reₒ).

The inventors of the present invention have demonstrated the existence of a platelet release phenomenon from a fluid F containing megakaryocytic cells in turbulent flow regimes and, also, a significant improvement in the platelet release phenomenon in certain very specific areas of the Andereck diagram.

These flow regimes (“spiral turbulence” or “Featureless turbulence” on the Andereck diagram) for Reynolds numbers of the inner cylinder

$${Re}_{i} = \frac{{r}_{i} \cdot {ω}_{i} \cdot d}{ϑ}$$

advantageously greater than 500 and of the outer cylinder

$${Re}_{o} = \frac{{r}_{o} \cdot {ω}_{o} \cdot d}{ϑ}$$

advantageously greater than 1000 in absolute value, where rᵢ is the radius of the inner cylinder 11, rₒ is the radius of the outer cylinder 13, ωᵢ is the angular speed of the inner cylinder 11, ωₒ is the angular speed of the outer cylinder 13, d is a distance separating the inner cylinder 11 from the outer cylinder 13 and V is the kinematic viscosity of the fluid between said inner and outer cylinders. This regime is obtained by rotating the cylinders in opposite directions around their axes (cylinder axles). These are known as contra-rotating cylinders.

Another of these regimes corresponds to a flow of the turbulent Taylor vortices (“Turbulent Taylor vortices” in the Andereck diagram), which we will refer to as “vortexes” in the following. This regime corresponds to a configuration in which the outer cylinder is stationary while the inner cylinder rotates around its axis. To achieve the regime of the Taylor turbulent vortices, the Reynolds number of the inner cylinder

$${Re}_{i} = \frac{{r}_{i} \cdot {ω}_{i} \cdot d}{ϑ}$$

is advantageously greater than or equal to 1400, where rᵢ is the radius of the inner cylinder 11, ωᵢ is the angular speed of the inner cylinder 11, d is a distance separating the inner cylinder 11 from the outer cylinder 13 and V is the kinematic viscosity of the fluid between said inner and outer cylinders.

These are the regimes for which the inventors have demonstrated an improvement in the platelet release process with a system of two circular concentric cylinders, it being understood that the release of the platelets can take place as soon as the flow regime is turbulent and therefore for ranges of Reᵢ and Reₒ greater than the aforementioned ranges.

As will become apparent from the following sections, the inventors of the present invention have demonstrated that the release of the platelets can be achieved not only by means of a system with two circular concentric cylinders, but also by means of other systems, all of which have in common with the above-mentioned system that two substantially parallel walls are separated by a predetermined distance obtained by experiment.

Referring to FIGS. 1a and 1b, an example of embodiment of the system 1 comprises two concentric cylinders 11, 13 separated by a space 15 with no mechanical parts. The system 1 comprises an inner cylinder 11 and a hollow outer cylinder 13 located radially external to the inner cylinder 11, said inner and outer cylinders 11, 13 being concentric. “Concentric cylinders” means that the inner and outer cylinders 11, 13 are coaxial, i.e. the axes of the inner and outer cylinders 11, 13 coincide.

The inner cylinder 11 corresponds to a solid comprising two parallel bases 11a, 11b, of substantially circular shape, separated by a distance, h, corresponding to the height of the inner cylinder 11. The inner cylinder 11 defines an outer lateral surface along its height h, which will be referred to hereafter as the “first wall 12”. Incidentally, the first wall 12 has a cylindrical shape.

The outer cylinder 13 is a hollow solid comprising a base 13a, substantially circular in shape, from which extends a second wall 14 which, similarly to the first wall 12, is cylindrical in shape. In this way, the outer cylinder 13 delimits a cylindrical housing 16 delimited externally by the base 13a and the second cylindrical wall 14 and having an opening at the level of its upper face, in the illustrated embodiment. Here, the terms “upper” and “lower” are not intended to be limiting and serve only to facilitate understanding of the invention with reference to the figures.

The housing 16 accommodates the inner cylinder 11 so as to allow the inner cylinder 11 to be arranged concentrically with the outer cylinder 13, as previously indicated. However, the inner cylinder 11 does not occupy the entire housing 16, as part of the housing 16 is left empty to form a space 15. The space 15 separates the first cylindrical wall 12 from the second cylindrical wall 14 and, more broadly, the inner cylinder 11 from the outer cylinder 13, so that the space 15 has the shape of a hollow cylinder delimited internally by the inner cylinder 11 and externally by the outer cylinder 13. The space 15 thus forms a reservoir allowing to accommodate the fluid F which, as a reminder, comprises the megakaryocyte cells that are precursors of the platelets. In addition, according to the invention, the space 15 has no mechanical parts. The advantages of this configuration are described below.

As can be seen from the previous section, the first cylindrical wall 12 is separated from the second wall 14 by a constant distance d. Similarly, the base 11a of the first cylinder, which is parallel to the base 13a of the outer cylinder 13, is also separated from the latter by a distance d′, which is not necessarily identical to the distance d. In the remainder of this description, the distance d separating the first cylindrical wall 12 from the second wall 14 is referred to as the “air gap”. The air gap is set when the system 1 is designed. The air gap is constant in order to control the flow regime in space 15. This being said, if the air gap is set by the dimensions of the first 11 and second 13 cylinders, the optimum air gap is not chosen as a function of said dimensions and, on the contrary, the dimensions of said inner and outer cylinders 11, 13 are chosen as a function of the desired air gap (but not solely). A significant improvement in the efficiency of the platelet release from the fluid F was demonstrated, depending on the air gap chosen and, moreover, on the speed or speeds of the first 12 and of the second 14 cylindrical walls.

It should be noted that the speed or the speeds of rotation v of the cylinder or cylinders depends on the dimensions of said inner and outer cylinders 11, 13. The linear speed v of each of said inner and outer cylinders 11, 13 is by definition dependent on the dimensions, and more particularly on each of the diameters, of said cylinders. So, while the dimensions of the inner and outer cylinders 11, 13 are chosen according to the desired air gap, they can also be selected taking into account the speed of rotation of the inner and outer cylinders 11, 13. For example, if the person skilled in the art is limited in his choice of the system for driving the cylinders, particularly in the drive power, he will have to take this additional constraint into account when designing the system 1.

The system 1 is equipped with a frame, not shown in FIGS. 1a 1b (but visible in FIG. 9), which allows said first 11 and second 13 cylinders to be arranged as described above. So, although the inner cylinder 11 appears to be suspended in mid-air in FIGS. 1a and 1b, it is actually held in its intended position by the frame. The same applies to the outer cylinder 13.

In practice, when the inner and outer cylinders 11, 13 move, they allow the fluid F contained in the space 15 to move. In this respect, the system 1 is also equipped with a system (not shown) for driving the inner and outer cylinders 11, 13. The drive system comprises a power supply system, one or more motors and a speed control system to control the motor or the motors. The drive system allows a defined speed to be applied independently to each of said cylinders 11, 13. It is then possible to implement the method according to the invention in different embodiments, which will be described in more detail below. For example, the drive system may consist of a magnetic drive system rotated by an electric motor controlled by a speed regulation system.

According to a first embodiment of the method according to the invention, the space 15 is filled with the fluid F comprising the megakaryocytes, then the inner cylinder 11 and the outer cylinder 13 are moved in opposite directions about their axes, so as to generate a turbulent flow of the fluid F in the space 15. In other words, the inner and outer cylinders 11, 13 perform rotational movements, the direction of rotation of the inner cylinder 11 being reversed with respect to the direction of rotation of the outer cylinder 13.

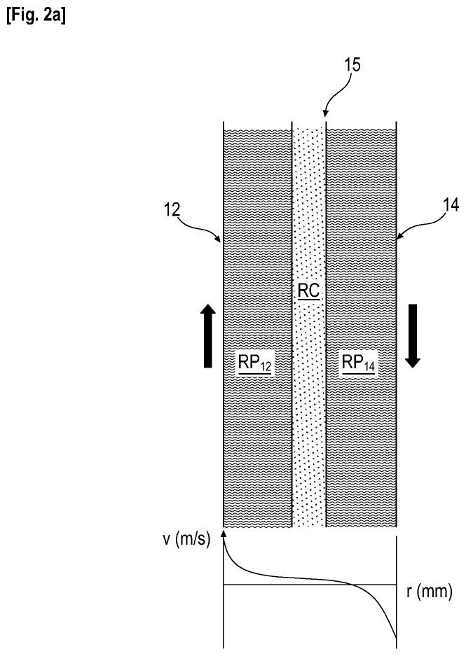

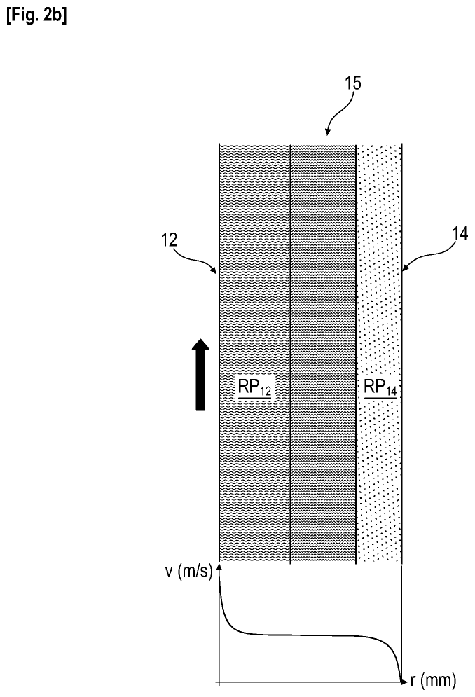

FIG. 2a shows the profile of the speeds within the space 15 when the inner and outer cylinders 11, 13 are moving in opposite directions. The system 1 as shown in FIG. 2a is a close top view of the system 1 as shown in FIGS. 1a and 1b. The first and second walls 12, 14 appear flat, whereas they are cylindrical. Furthermore, the system 1 as shown is not to scale and the dimensions are chosen only to allow a better illustration of the local flow regimes in the space 15. The flow speed of the fluid F, denoted v in the following, is relatively high in the vicinity of each of the walls 12, 14, i.e. at the level of the areas RP₁₂ and RP₁₄ respectively, and a speed v of the fluid F of almost zero in a central region RC of the space 15. The flow of the fluid F in this central region RC and in the areas RP₁₂ and RP₁₄ is turbulent. So, while on average the fluid F is static radially between the first wall 12 and the second wall 14, there is significant turbulence in the regions close to the walls RP₁₂ and RP₁₄.

Advantageously, a turbulent flow is generated by raising the inner cylinder 11 and the outer cylinder 13 respectively to Reynolds numbers greater than 1000. That said, this is a preferred mode and not a prerequisite for obtaining the release of the platelets.

Advantageously, said inner and outer cylinders 11, 13 are separated by an air gap d of less than 5 mm, but not zero due to the presence of the space 15. Within this range of values (0 mm<d≤5 mm), it is possible to improve the platelet release efficiency whereas outside this range, particularly when d>5 mm, the platelet release efficiency is very low. Thus, compared to a reference sample for which the release of the platelets is very low, the air gap values in the range 0 mm<d≤5 mm allow to improve the platelet release efficiency.

In practice, the air gap d is associated with a speed of rotation v of said inner and outer cylinders 11, 13, so that the system 1 can advantageously be defined by a given pair (d, v). The speed of rotation of a cylinder is itself linked to the Reynolds number of the cylinder. Each geometry (i.e. dimensions) of the system 1 is associated with one or more optimal pairs (d, v), i.e. which allow to generate a turbulent flow that allows to optimise the release of the platelets from the fluid F. However, while an optimal speed of rotation v depends intrinsically on the geometry of the system 1, the optimal air gap does not depend on this geometry. Therefore, as mentioned above, the optimum air gap d allowing to achieve the highest platelet release efficiency was determined empirically, and the geometry of the system 1 is then chosen to take account of the air gap. It is therefore clear that there are many different geometries for system 1, as illustrated in FIGS. 1a and 1b, which allow this constraint to be met. The choice of a geometry will also take into account the type of system for driving the inner and outer cylinders 11, 13 that is available. A person wishing to manufacture the system 1 and having a drive system that only allows him to rotate a small system will be limited by the volumes of fluid F treated.

According to one example of embodiment of the system 1, the inner cylinder 11 has an outside diameter of between 2 and 6 cm, the outer cylinder 13 has an inside diameter of between 2 and 6 cm and the outer cylinder 13 has a height h of between 5 and 6 cm. Such a system 1 allows to achieve an improved platelet release efficiency with air gaps in the range 0 mm<d≤5 mm, as previously mentioned, and for a configuration in which the inner and outer cylinders 11, 13 move in opposite directions at a rotational speed v of between 0.5 m/s and 2 m/s. For example, a system 1 in which the inner cylinder 11 has an outer diameter of 27 mm, the outer cylinder 13 has an inner diameter of 33 mm and the outer cylinder 13 has a height of 60 mm allows to achieve an improved platelet release efficiency when the air gap is in the range 0 mm<d≤5 mm and the inner and outer cylinders 11, 13 move in opposite directions at a rotational speed v of between 0.8 m/s and 1.5 m/s respectively.

Even more advantageously, said first and second cylinders 11, 13 are separated by an air gap d of between 2 mm and 4 mm. This configuration is advantageous in that the platelet release efficiency is further improved when the method is implemented compared with a method implemented using a system 1 with an air gap outside this range. Taking the example of a system 1 in which the inner cylinder 11 has an outside diameter of between 2 and 6 cm, the outer cylinder 13 has an inside diameter of between 2 and 6 cm and the outer cylinder 13 has a height h of between 5 and 6 cm, the optimum speeds of rotation v of the inner and outer cylinders 11, 13 are between 0.9 m/s and 1.2 m/s, said cylinders moving in opposite directions respectively.

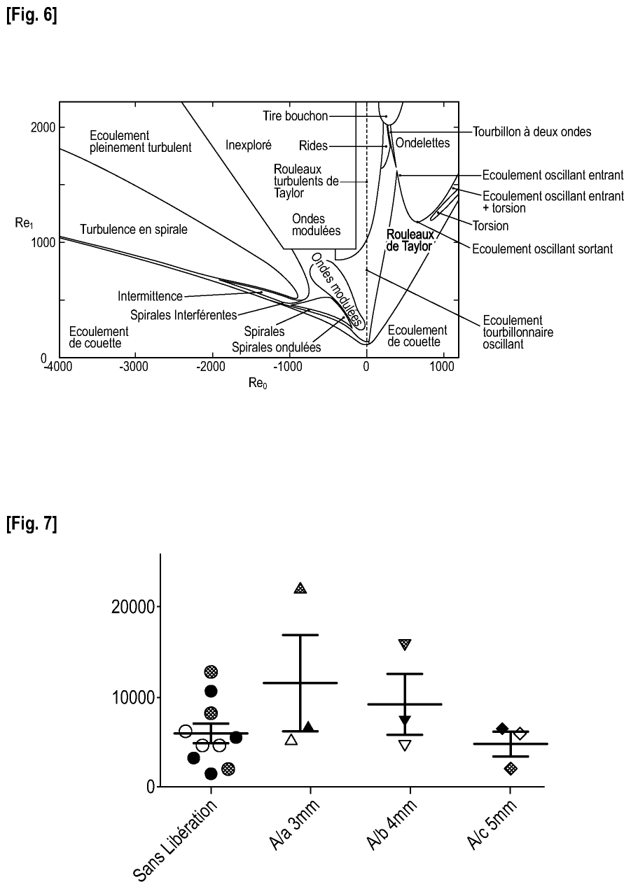

Very advantageously, said first and second cylinders 11, 13 are separated by an air gap d of 3 mm. This air gap allows to obtain a better platelet release efficiency in the configuration illustrated in FIGS. 1a and 1b compared with the case where the method according to the invention is implemented with an air gap outside this range. FIG. 7 illustrates such an optimum in the configuration where the two cylinders rotate in opposite directions. Taking the example of a system 1 in which the inner cylinder 11 has an outside diameter of between 2 and 6 cm, the outer cylinder 13 has an inside diameter of between 2 and 6 cm and the outer cylinder 13 has a height h of between 5 and 6 cm, the optimum speed of rotation v of the inner and outer cylinders 11, 13 is approximately 1 m/s respectively, said cylinders moving in opposite directions.

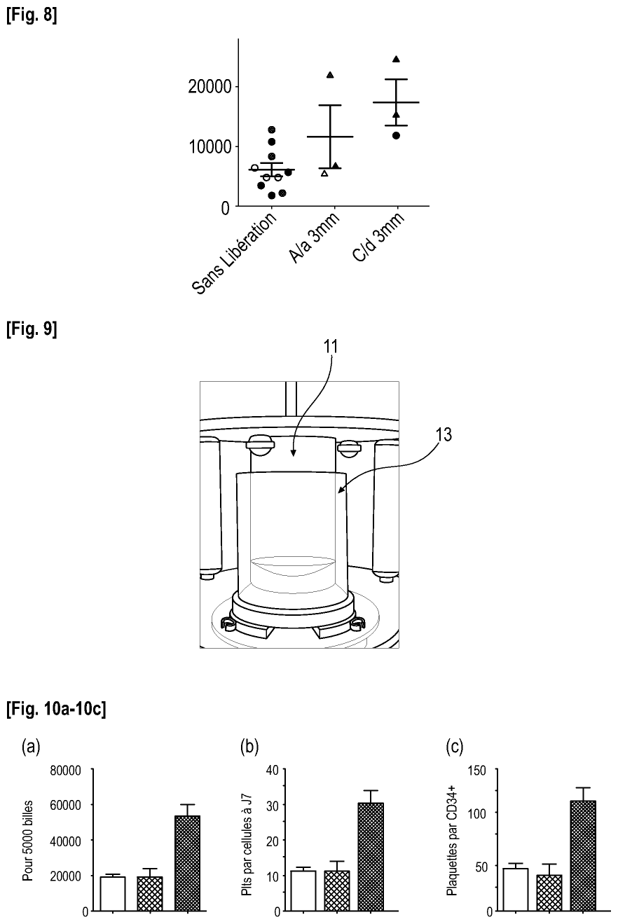

According to a variant of the first embodiment of the method according to the invention, a first step of the method consists in filling the space 15 with the fluid F and a second step of the method consists in rotating the first wall 12, while the second wall 14 remains stationary, it being understood that the inner cylinder 11 is rotated about its axis while the outer cylinder 13 remains stationary. Under these conditions, a turbulent flow regime is obtained in the space 15. FIG. 8 shows that platelet release efficiency is further improved when only the inner cylinder 11 rotates around its axis (right-hand sample) compared to the configuration where both cylinders 11, 13 rotate in opposite directions.

FIG. 2b shows the profile of the speeds of the fluid F between the first wall 12 and the second wall 14 when only the first wall 12 is rotating. The system 1 as shown in FIG. 2b is a close top view of the system 1 as shown in FIGS. 1a and 1b. As shown in the diagram, when only the inner cylinder 11 is rotating, the inventors have observed a gradual decrease in the speed v of the fluid F as it moves away from the first wall 12. In other words, the speed v of the fluid F is highest at the level of the first wall 12 and then decreases progressively as it approaches the second wall 14, to the point where it is very low at the level of the second wall 14. This speed profile differs substantially from that seen for the configuration in which the two cylinders 11, 13 are in counter-rotation. A circulation of the fluid as described below can be used to compensate for this radial variation in the speed.

Advantageously, a fully turbulent flow is generated, allowing to generate turbulent vortices that allow to increase the platelet release. For this flow regime to be achieved by means of the system 1, the inner cylinder 11, which alone rotates about its axis, must be driven at a very high angular speed corresponding to a Reynolds number of the inner cylinder 11 greater than 1400. This flow regime is characterised by a low-turbulence flow in which there are vortices, highly turbulent phenomena, also known as “rollers” in the scientific literature, around the axis of rotation of the cylinders, along the height of the cylinders.

As in the first embodiment, in which the two cylinders 11, 13 rotate, the air gap is advantageously in the range 0 mm<d≤5 mm, even more advantageously 2 mm≤d≤4 mm, and very advantageously d=3 mm. The same effects were observed as in the first embodiment. That said, the optimum rotation speeds v associated with each of these preferred implementations differ substantially in that only the inner cylinder 11 rotates.

Consider a system 1 in which the inner cylinder 11 has an outer diameter of between 2 and 6 cm, the outer cylinder 13 has an inner diameter of between 2 and 6 cm and the outer cylinder 13 has a height h of between 5 and 6 cm. In such a system 1, the optimum speed of rotation v of the inner cylinder 11 is between 0.8 m/s and 2.5 m/s for an air gap d between 0 mm (0 mm not included) and 5 mm, between 1.2 m/s and 2.2 m/s for an air gap d between 2 mm and 4 mm and between 1.6 m/s and 1.9 m/s for an air gap of approximately 3 mm.

Furthermore, if the occurrence of a regime of flow of the fluid F in the space 15 allows platelets to be released, it is also important that the platelets produced behave like in vivo platelets in terms of their biochemical activity. This is achieved by the absence of any mechanical parts in the space 15, which contains only the fluid F. Unlike the systems known in the prior art in which a moving blade in the reservoir containing the fluid allows to create the agitation within said reservoir, in the invention, the turbulence is generated by the speed of movement of the inner cylinder 11 and, where appropriate, the outer cylinder 13. This absence of moving mechanical parts in the space 15 allows to prevent any collisions with the megakaryocytes, thereby allowing to preserve the biochemical activity of the released platelets. An inactive platelet, or one whose activity is reduced, cannot take part in the mechanisms for preserving the structural integrity of the blood vessels, in the primary haemostasis or in mechanisms such as the pro-coagulation. Similarly, it allows to preserve the biochemical activity of the extracellular vesicles released from the platelets.

The system 1 illustrated in FIGS. 1a and 1b can have dimensions ranging from a few centimetres to several tens of centimetres. In order to increase the volume of fluid treated while maintaining a high platelet release efficiency, one solution is to increase the size of the inner and outer cylinders 11, 13. That said, the implementation of this solution in the configurations described above requires a significant increase in the dimensions of the inner and outer cylinders 11, 13 in order to maintain an air gap of between 0 mm (0 mm not included) and 5 mm.

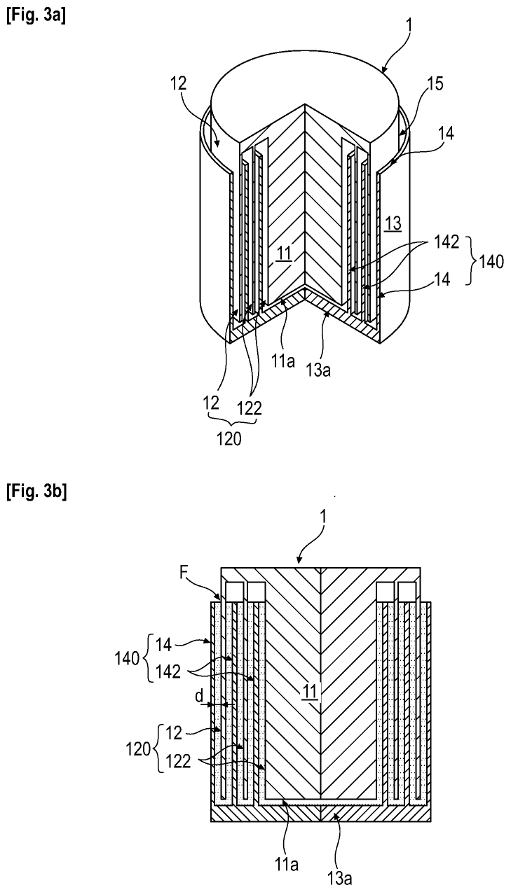

It is in this context that the inventors of the present invention have implemented the system 1 as illustrated in FIGS. 3a and 3b. In this embodiment, the system 1 comprises an inner cylinder 11 and an outer cylinder 13 such as the system 1 illustrated in FIGS. 1a and 1b and differs therefrom only in the following characteristics.

As illustrated in FIGS. 3a and 3b, the inner cylinder 11 and the outer cylinder 13 each comprise further walls 122, 142. In particular, walls 122, 142 are added to the first wall 12 and to the second wall 14 of said cylinders respectively. In other words, the inner cylinder 11 comprises the first wall 12 and other walls 122, while the outer cylinder 12 comprises the second wall 14 and other walls 142. The first wall 12 and the other walls 122 form a first pattern 120, while the second wall 14 and the other walls 142 form a second pattern 140.

The first pattern 120 and the second pattern 140 are nested so that the first wall 12 and the other walls 122 on the one hand and the second wall 14 and the other walls 142 on the other hand are parallel to each other and at least partially opposite each other. The system 1 thus obtained comprises an alternation of parallel cylindrical walls, the outermost wall being the second wall 14, then comes the first wall 12, then a first other wall 142, then a first other wall 122, then a second other wall 142 and so on. The first wall 12 is therefore at least partially opposite the second wall 14 and the first other wall 142. The expression “at least partially opposite” is intended to mean that the first wall 12 is not opposite said second wall 14 and said first other wall 142 over its entire height. As in the configuration illustrated in FIGS. 1a and 1b, an air gap d′ exists between the base 13a of the outer cylinder 13 and the “base” 11a of the inner cylinder 11. However, it should be noted that the base 11a of the system 1 as illustrated in FIGS. 3a and 3b is not continuous, since there are empty spaces between the walls which are left for the fluid F to penetrate. The base 11a of the inner cylinder 11 is therefore discontinuous in this configuration.

This configuration is particularly advantageous because it allows the volume of fluid F treated to be increased without excessively increasing the dimensions of the system 1. Alternating the walls 12, 14, 122, 142 create numerous intermediate spaces between a central axis of the system 1 and the second wall 14. These are all spaces into which the fluid F can enter. In the example of embodiment shown in FIGS. 3a and 3b, the system 1 comprises a first wall 12 and two other walls 122 and a second wall 14 and two other walls 142. This means that 4 additional spaces have been created, extending around the circumference. The number of other walls is not limited to the example of embodiment shown in FIGS. 3a and 3b. Other walls 122, 142 could thus be provided, it being understood that if there are n, n being a strictly positive natural number, other walls 122 and n other walls 142, there will be 2×n additional spaces, i.e. in addition to the space 15. Incidentally, the larger the diameter of said inner and outer cylinders 11, 13, the more additional spaces can be created and the greater the volume of fluid treated.

Preferably, the air gap is adapted between two successive spaces of the system 1, for all the spaces formed between the walls 12, 14, 122 and 142 so as to maintain the same Reynolds in the different spaces of the system 1. This is because the angular speed of rotation is constant radially, the linear speed varies from one space 15 to another and it is preferable to adapt the air gap so that the platelet release efficiency is improved uniformly throughout the system 1. The air gap d between the second wall 14 and the first wall 12 is therefore different from the air gap d between the first wall 12 and the first other wall 142 and the air gap d between the first other wall 142 and the first other wall 122, and so on.

The optimum pairs (d,v), i.e. those that allow to generate a turbulent flow allowing to optimise the release of the platelets from the fluid F, are the same as those previously seen. At this stage, it should be pointed out that the method according to the invention can be implemented according to the two previously described embodiment with a system 1 in the configuration illustrated schematically in FIGS. 3a and 3b. It is therefore just as possible to rotate the inner and outer cylinders 11, 13 in opposite directions as to rotate only the inner cylinder 11. Whatever the configuration considered, the optimum pairs (d,v) are the same and the platelet release efficiencies obtained are similar. Note that the configuration illustrated in FIGS. 3a and 3b is advantageous compared with that illustrated in FIGS. 1a and 1b in that the volume of fluid F treated can be significantly increased.

In addition to the air gap d and the speed of rotation v of the inner and outer cylinder or cylinders 11, 13, it is also possible to influence the platelet release phenomenon by means of a third parameter, which is the residence time of the fluid in the reservoir. As a reminder, in the system 1 as exemplified in FIGS. 1a and 1b, the space 15 forms a reservoir for the fluid F, whereas in the system 1 as exemplified in FIGS. 3a and 3b it is no longer just the space 15 that forms a reservoir for the fluid F, but also all the spaces created between the parallel cylindrical walls. To vary the residence time of the fluid in either case, the system 1 can be equipped with an inlet orifice (not shown in the figures) at the level of the frame and an outlet orifice (not shown in the figures) at the level of the base 13a of the outer cylinder, allowing the fluid F to penetrate and leave the reservoir respectively.

In addition, when the device is equipped with an inlet orifice and an outlet orifice, it is possible to set up a process for circulating the fluid F within the reservoir whatever the configuration envisaged, i.e. whether the two inner and outer cylinders 11, 13 rotate in opposite directions or whether only the inner cylinder 11 rotates. The circulation of the fluid involves continuously recovering the fluid that has already been treated and removed from the reservoir, and replacing it with the untreated fluid from a reservoir. Of course, such circulation is advantageously performed in a closed loop so that the fluid F is never exposed to the external medium and to the sources of contamination. The circulation therefore allows to transform the batch treatment method into a continuous treatment method. This is particularly advantageous for an industrial use.

The residence time of the fluid is more particularly controlled by acting on the opening diameter, or more generally on the opening cross-section of the outlet orifice. This represents a further step towards the industrialisation of the method according to the invention, since it then becomes possible to continuously treat the fluid F comprising megakaryocytes. In this configuration, the dimensions of the system 1 are of secondary importance, since the fluid F is treated continuously, so that several litres of fluid can be treated in just 1 hour. That said, the residence time of the fluid must be selected so as to optimise the platelet release efficiency.

Advantageously, the residence time of the fluid F within the reservoir is between 4 minutes and 6 minutes, and preferably around 5 minutes. When the residence time of the fluid in the reservoir is less than 5 minutes, the platelet release efficiency is not optimal. However, when the residence time of the fluid in the reservoir exceeds 5 minutes, no additional benefit is observed. In other words, the platelet release efficiency does not increase because the fluid F remains in the reservoir for longer than 5 minutes. It is therefore not necessary to exceed a residence time of 5 minutes. This was observed for any of the implementations of the method previously described, namely with the inner and outer cylinders 11, 13 rotating in opposite directions in the system 1 illustrated in FIGS. 1a and 1b or that illustrated in FIGS. 3a and 3b as well as with the inner cylinder 11 alone rotating in the system 1 illustrated in FIGS. 1a and 1b or that illustrated in FIGS. 3a and 3b.

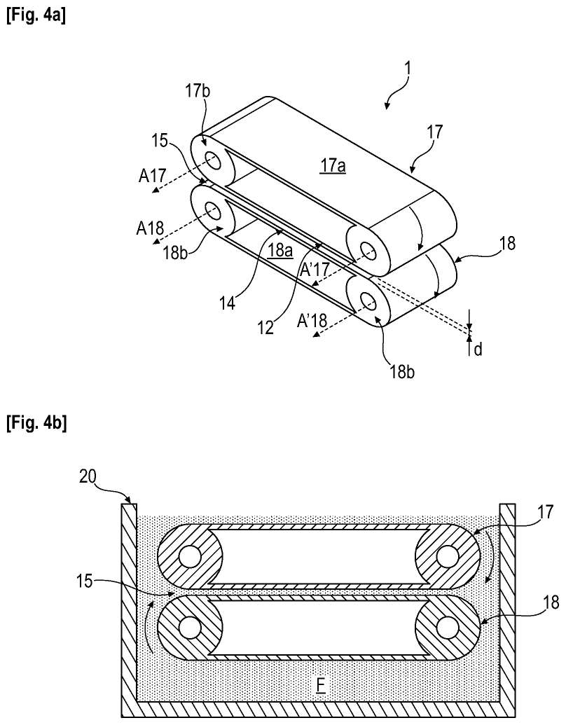

Reference is made to FIGS. 4a and 4b. In a second embodiment of the method according to the invention, it is implemented by means of a system 1 comprising two conveyor belts 17, 18. The conveyor belts 17, 18 each comprise an endless strip 17a, 18a closely arranged around two guide means 17b, 18b. The guide means 17b, 18b are roller-shaped. They are separated by a sufficient distance for the endless strip 17a, 18a to be tensioned so that the endless strip perfectly matches the outer contours of the guide means 17b, 18b on lateral edges of each of the conveyor belts 17, 18 and so that, outside the areas where the endless strip matches the outer contours of the guide means, the endless strip 17a, 18a has relatively large flat surfaces. By “large dimension” we mean flat surfaces of a few centimetres to a few tens of centimetres.

A first conveyor belt 17 referred to as the “upper belt” comprises a first flat wall 12, corresponding to one of the flat surfaces of said upper belt 17, while a second conveyor belt 18 referred to as the “lower belt” comprises a second flat wall 14, corresponding to one of the flat surfaces of said lower belt 18. The upper belt 17 and the lower belt 18 are superimposed. In other words, the upper belt 17 is positioned opposite the lower belt 18. It is also arranged parallel to the lower belt 18 so that, as in the first embodiment (FIGS. 1a to 3b), the first wall 12 and the second wall 14 are parallel and at least partially opposite each other. It should be noted that the terms “upper” and “lower” are not intended to be restrictive and serve only to facilitate the understanding of the invention with reference to the figures.

The upper belt 17 is separated from the lower belt by a non-zero distance d, so that a space 15 is formed between the two belts. The space 15 located between the upper belt 17 and the lower belt 18 is similar to the space 15 located between the inner cylinder 11 and the outer cylinder 13 in the first embodiment of the system 1. However, although as in the first embodiment it can allow to accommodate the fluid F, unlike the first embodiment it does not form a closed reservoir for the fluid F. In fact, in the embodiment illustrated in FIGS. 4a and 4b, the system 1 comprises a reservoir 20 in which the conveyor belts 17, 18 are arranged. This reservoir contains the fluid F as described above. The system I also comprises a frame (not shown) which allows the conveyor belts 17, 18 to adopt the position shown in FIGS. 4a and 4b.

The endless strips 17a, 18a are able to move. In this respect, the guide means 17b, 18b will guide in rotation by means of a drive system about their respective axes of rotation A17, A′17, A18, A′18. Like the drive system of the first embodiment, it comprises a power supply system, one or more motors and a speed regulation system used to control the motor or motors. The drive system allows a defined speed to be applied independently to each of said guide means 17b, 18b. The guide means 17b, 18b can rotate in either direction about their respective axis of rotation and consequently cause the endless strips 17a, 18a to move in a given direction.

Thus, the method according to the invention can be implemented either by rotating the endless strips 17a, 18a in opposite directions, each of said first and second walls 12, 14 being moved in its plane, or by rotating only one of the endless strips 17a, 18a, one of the first and second walls 12, 14 being moved in its plane. The person skilled in the art will then understand that, as in the first embodiment, it is thus possible to generate a turbulent flow regime in the space 15 favourable to the release of platelets from the megakaryocytes contained in the fluid F. To further improve the platelet release efficiency, it is then sufficient to choose an optimum air gap d. At the same time, it is sufficient to choose a speed of rotation v of the guide means in the appropriate range, i.e. in the speed range where the regime of flow of the fluid F allows to optimise the release of the platelets. The speed of rotation is chosen so as to achieve a turbulent flow regime and taking due account of the dimensions of the conveyor belts 17, 18 since the speed of rotation depends on this parameter.

Advantageously, the upper conveyor belt 17 and the lower conveyor belt 18 move at the same speed and in opposite directions. In this configuration, the fluid is even more sheared even though it remains static on average between the first wall 12 and the second wall 14. In addition, in this configuration, the optimum residence time would then be the same as in the embodiment with the inner and outer cylinders 11, 13, i.e. a residence time of between 4 minutes and 6 minutes, preferably around 5 minutes, would be sufficient to obtain an optimum platelet release efficiency. That said, this is not compulsory, since a difference in rotation speed between the conveyor belts 17, 18 means that a continuous supply of fluid F can be envisaged.

Alternatively, the upper conveyor belt 17 and the lower conveyor belt 18, and therefore the two walls 12, 14, move with a speed difference of at most 10% with respect to an average value. An appropriate shear of the fluid is maintained while retaining flexibility in the design of the device 1.

The system 1 in this second embodiment operates in a similar way to the system 1 in the first embodiment, except that the Reynolds numbers of the conveyor belts are calculated differently. Indeed, when the diameter of a cylinder approaches very large values or tends towards infinity, the cylindrical wall of this cylinder can be assimilated to a flat wall. For example, considering a system 1 used in the first embodiment, where it is known that the dimensions of the cylinders 11, 13 vary between a few centimetres and a few tens of centimetres, a very large cylindrical wall would have a diameter of the order of a metre at least. Thus, locally such a wall could be considered flat. In the second embodiment of the system 1, the first wall 12 and the second wall 14 of the conveyor belts 17, 18 can be likened to the walls of very large diameter cylinders. This explains why the phenomena involved in the turbulent flows seen in relation to the system 1 according to the first embodiment, in the configuration where the two cylinders 11, 13 rotate and in the configuration where only the inner cylinder 11 rotates, can be reproduced by means of the system 1 with two conveyor belts.

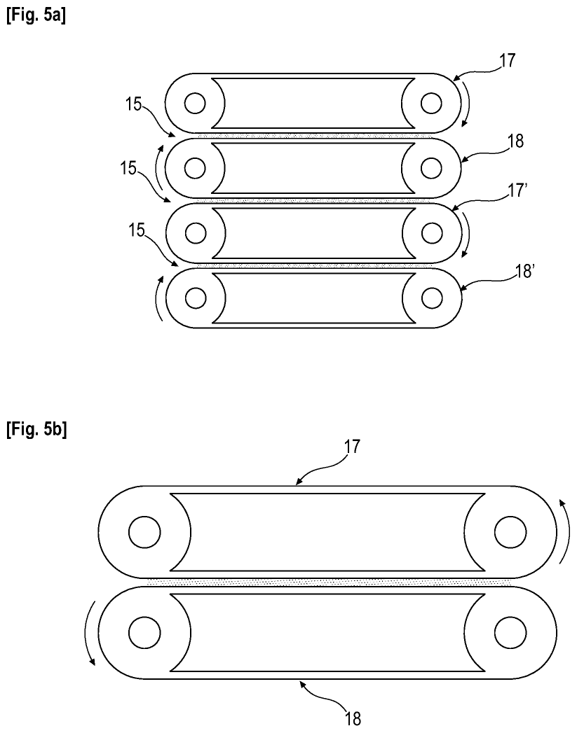

In addition, as shown in FIG. 5a, it is also possible to provide more conveyor belts. In the variant shown in FIG. 5a, an additional pair of conveyor belts 17′, 18′ is provided in the system 1. They are superimposed on the upper and lower conveyor belts 17, 18 and therefore allow to obtain a system 1 comprising four conveyor belts and three spaces 15. Each of these spaces 15 constitutes a turbulent flow area. The number of conveyor belts is not limitative and a greater number of belts could be imagined, provided however that the dimensions of the reservoir 20 are appropriate to accommodate the number of conveyor belts envisaged. The greater the number of conveyor belts, the greater the volume of fluid F that can be treated. This example of embodiment should be compared with the example of embodiment of the system 1 shown in FIGS. 3a and 3b, in which the space 15 has been considerably extended by a set of other walls 122, 142 in each of the cylinders.

In the example of embodiment shown in FIG. 5b, the upper and lower conveyor belts 17, 18 are larger. This allows to increase the length of the space 15 and therefore the volume of fluid F treated. However, this option may be more restrictive than the previous one insofar as the motorisation associated with larger guide means 17b, 18b may also present technological and/or economic constraints.

### Materials and Methods of the Present Invention

The method according to the invention has been implemented using the device illustrated in FIG. 9 and experimental results are illustrated in the following figures and commented on in the following sections.

### Differentiation of the Megakaryocytes in Culture

The haematopoietic progenitors CD34⁺ are extracted from leucodepletion filters (TACSI, Terumo BCT, Zaventem, Belgium) used for the preparation of labile blood products. The cells contained in the filter are eluted and enriched in cells CD34⁺ by magnetic sorting using antibodies anti-CD34⁺ coupled to magnetic beads (CD34 MicroBead Kit UltraPure, Miltenyi Biotec, Bergisch Gladbach, Germany). The cells were then seeded in the StemSpan serum-free expansion medium (SFEM) supplemented with 20 μg/mL human low density lipoprotein (LDL), a cocktail of cytokines (IL-6, IL-9, SCF and TPO) (CC220, Stemcell Technologies, Vancouver, BC, Canada) and by 1 μM SR1 (Stemcell Technologies). On day 7, the cells are harvested, washed, seeded in the StemSpan SFEM containing 1 μM SR1, 50 ng/ml of TPO and 20 μg/mL of LDL and cultured for a further 6 days (Strassel et al., 2016). The cultures were incubated at 37° C. under normoxic conditions and a 5% of CO₂ atmosphere.

### Release of the Culture Platelets

After 13 days in culture, the megakaryocytes show cytoplasmic extensions referred to as proplatelets. The medium containing the proplatelet megakaryocytes is placed in the Couette device and the platelets are released in one of the configurations defined below.

Two configurations of the Couette device were tested:
Configuration “2 Rotors” where the two rotors (inner cylinder 11 and outer cylinder 13) of the device rotate in opposite directions
Configuration “Rotor Up” where only the inner rotor 11 rotates.

These configurations were determined on the basis of the mapping of the flows studied for a fluid between two closely spaced walls moving relative to each other.

The number of platelets released is estimated using tubes containing calibration beads (BD Trucount™, BD Biosciences, San Jose, USA). The platelet count is expressed for the passage of 5,000 beads and then reduced to the number of platelets per cell at D7 and the number of CD34⁺ cells seeded at D0.

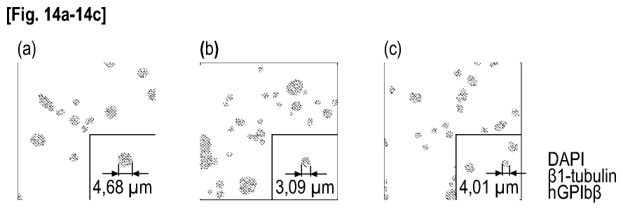

### Study of the Morphology (FIG. 14)

After fixation in the paraformaldehyde, the platelets were cytospinned, permeabilised with 0.1% of Triton X-100 and incubated with an anti-β1-tubulin antibody (1 μg/mL, Eurogentec, Liège, Belgium) followed by a GAM-488 secondary antibody (10 μg/mL) and an anti-GPIIbIIIa mAb (10 μg/mL), diluted in the PBS 1% of BSA (Bovin Serum Albumin). The cells were then incorporated in the Mowiol (Mountant, Permafluor, Thermo Fisher Scientific, UK) and examined using a confocal microscope (TCS SP8, Leica Microsystems, Rueil-Malmaison, France) equipped with an oil objective (F-type immersion liquid, ne23=1.5180, ve=46, Leica Microsystems). The data were acquired using the LASAF software, version 1.62 (Leica Microsystems).

### Study of the Functionality In Vitro

### Activation Capacity (FIG. 12)

For the activation assays, the platelet suspension is labelled with the antibodies directed against the GPIIb-IIIa and against the GPIbα and incubated for 10 minutes at room temperature. Three analysis tubes are produced, containing either: anti-CD62P antibody, PAC1 antibody (anti GPIIb-IIIa in its activated form), or the isotopic control. After 10 minutes incubation at room temperature, 10 μg/mL of 10 μM TRAP (for the activated tubes) or PBS (for the non-activated tubes) was added. The percentage of activated platelets was analysed by flow cytometry.

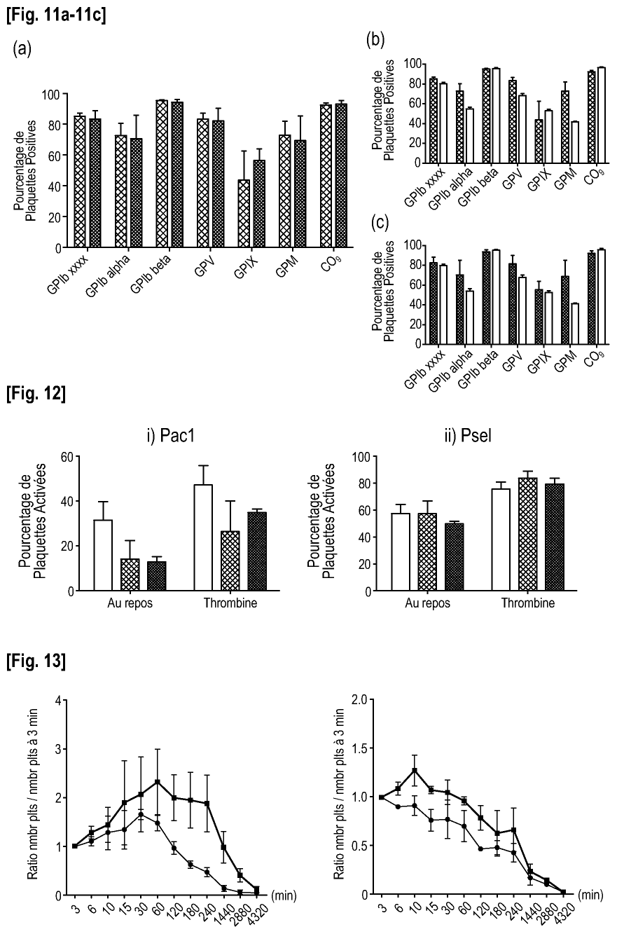

### Glycoprotein Expression (FIG. 11)

For the expression assays of the glycoproteins, the platelet suspension is labelled with the antibodies directed against GPIIb-IIIa, GPIbα, GPIbβ, GPV, GPIX, GPVI and CD9. After incubation for 30 minutes at room temperature, the samples were analysed by flow cytometry. FMOs (Fluorescence Minus One) are carried out to determine the analysis windows when using several fluorochromes.

### Study of the Functionality In Vivo (FIG. 13)

### Recirculation After Transfusion

The cultured platelets are harvested after addition of 0.5 μM illoprost (Ilomedine 0.1 mg/1 ml, Bayer AG, Germany) to the medium containing the proplatelet megakaryocytes. The platelets are then centrifuged and resuspended in the Tyrode's buffer containing albumin, as described in the literature (Hechler et al., 2013; Strassel et al., 2016). After the final washing step, the platelets are transferred to a preservation solution composed of one third plasma and two thirds additive solution, the Intersol (Fresenius Kabi, Homburg, Germany). This preservation solution is used for the storage and the transfusion of the native platelets.

Aliquots containing 5×10⁷ washed human platelets, cultured or native from MCP (mixed platelet concentrate), were injected via the retro-orbital vein into NSG mice (NOD.Cg-Prkdc scid, Il2rg tm 1 Wjl/SzJ) previously depleted of macrophages (by injection of clodronate liposomes on day −1).

The circulation of the human and mouse platelets was analysed by flow cytometry in the whole blood samples taken at 3, 6, 10, 15, 30, 120, 240, 1400, 2800 and 4320 minutes post-transfusion. Each blood sample is labelled with the RAM.1-568 antibody, recognising the human and mouse GPIbβ, and the ALMA17-488 antibody, recognising only the human GPIIb-IIIa, before being analysed by flow cytometry (Do Sacramento et al., 2020). In order to be able to compare the results, a ratio of the number of human platelets to the total number of platelets was established at the first stage of the analysis at 3 minutes, arbitrarily set at 1.

1. A method for releasing platelets from megakaryocytes contained in a fluid (F), the method being implemented by a system comprising two concentric cylinders, an inner cylinder comprising a cylindrical wall and a hollow outer cylinder situated radially external to the inner cylinder, the outer cylinder comprising a cylindrical wall of a base at a level of which the second cylinder is closed, the cylinders being separated by a space devoid of any mechanical part, the space being intended to receive the fluid (F), the method comprising the following steps:
supplying the space with a fluid (F) comprising megakaryocytes,
rotating the inner cylinder about its axis, the outer cylinder being stationary, or
moving the two cylinders in opposite directions about their axes, so as to generate an at least partially turbulent flow of fluid (F) in the space and obtain a second fluid (F′) enriched with platelets.

2. The method according to claim 1, wherein when the inner cylinder is moved while the outer cylinder is stationary, the inner cylinder is moved so as to define a Reynolds number
$${Re}_{i} = \frac{{r}_{i} \cdot {ω}_{i} \cdot d}{ϑ}$$
greater than or equal to 1400, where rᵢ is the radius of the inner cylinder, ωᵢ is the angular speed of the inner cylinder, d is a distance separating the inner cylinder from the outer cylinder and ϑ is the kinematic viscosity of the fluid between the inner and outer cylinders.

3. The method according to claim 1, wherein, when the two cylinders are moved in opposite directions, the inner cylinder and the outer cylinder are moved so as to define respectively a Reynolds number
$${Re}_{i} = \frac{{r}_{i} \cdot {ω}_{i} \cdot d}{ϑ}$$
greater than 1000 and a Reynolds number
$${Re}_{o} = \frac{{r}_{o} \cdot {ω}_{o} \cdot d}{ϑ}$$
greater than 1000, where rᵢ is the radius of the inner cylinder, rₒ is the radius of the outer cylinder, ωᵢ is the angular speed of the inner cylinder, ωₒ is the angular speed of the outer cylinder, d is a distance separating the inner cylinder from the outer cylinder and ϑ is the kinematic viscosity of the fluid between the inner and outer cylinders.

4. The method according to claim 1, wherein the two cylinders are separated by a distance (d) of less than 5 mm.

5. The method according to claim 1, wherein the two cylinders are separated by a distance (d) of between 2 mm and 4 mm.

6. The method according to claim 1, wherein the two cylinders are separated by a distance (d) of approximately 3 mm.

7. The method according to claim 1, wherein the space is continuously supplied with the fluid and the system comprises an inlet for filling the space with the fluid (F) and an opening for evacuating the fluid (F′) enriched with platelets, the opening being located at the level of the base.

8. The method according to claim 7, wherein the residence time of the fluid (F) in the space is between 4 minutes and 6 minutes.

9. The method according to claim 1, wherein the inner cylinder and the outer cylinder comprise other peripheral internal walls forming a first pattern and a second pattern respectively, the first and second patterns being nested in one another so that the other walls of the inner cylinder and other walls of the outer cylinder are concentric with one another and at least partially opposite one another.

10. A method for releasing platelets from megakaryocytes contained in a fluid (F), the method being implemented by a system comprising two parallel flat walls separated by a space devoid of any mechanical part, the space being designed to receive the fluid (F), the walls being suitable for movement, the method comprising the following steps:
supplying the space with a fluid (F) comprising megakaryocytes,
moving one of the flat walls in a plane of the flat wall, the other of the walls being stationary, or
moving the two walls in opposite directions, each wall being moved in its plane, so as to generate an at least partially turbulent flow of fluid (F) in the space and obtain a second fluid (F′) enriched with platelets.

11. The method according to claim 10, wherein the first flat wall is the wall of a first conveyor belt and the second flat wall is the wall of a second conveyor belt.

12. The method according to claim 10, wherein when the two walls are moving in opposite directions, the two walls are moving in opposite directions at the same speed.

13. The method according to claim 12, wherein the two walls move at a speed of approximately 1 metre per second.

14. The method according to claim 10, wherein when the two walls move in opposite directions, the two walls move with a speed difference of at most 10% with respect to an average value.

15. The method according to claim 10, wherein extracellular vesicles are additionally released.

16. The method according to claim 7, wherein the residence time of the fluid (F) in the space is 5 minutes.
